(19) United States
(12) Patent Application Publication
(10) Pub. No.: US 2008/0044805 A1
(43) Pub. Date: Feb. 21, 2008

(54) Biopolymer-Dye Complex and Use as a Bioassay

(75) Inventors: David G. Whitten, Albuquerque, NM (US); Komandoor E. Achyuthan, Santa Fe, NM (US); Gabriel P. Lopez, Albuquerque, NM (US)

Correspondence Address: GONZALES PATENT SERVICES, 4605 CONGRESS AVE. NW, ALBUQUERQUE, NM, 87114, US

(73) Assignee: STC.UNM, Albuquerque, NM (US)

(21) Appl. No.: 11/842,710

(22) Filed: Aug. 21, 2007

Related U.S. Application Data
(60) Provisional application No. 60/839,001, filed on Aug. 21, 2006.
(60) Provisional application No. 60/843,171, filed on Sep. 8, 2006.

(51) Int. Cl. G01N 33/00

(52) U.S. Cl. 435/4

(57) ABSTRACT

The present disclosure provides rapid, simple, robust and sensitive label and label-free assays for use in drug discovery, bioagent detection and medical diagnostics as well as means for reading such assays. The assays disclosed in the present disclosure use the formation, disruption and destruction of self-assembling fluorescent dye:biopolymer complexes to indicate the presence or absence of an analyte of interest. Such assays are suitable for high throughput screening and can achieve high sensitivity due to the enhanced fluorescence and changes of absorption spectra of certain dyes, particularly cyanine dyes, when they bind to specific biopolymers.

### CROSS-REFERENCE TO RELATED APPLICATIONS

The following application claims benefit of U.S. Provisional Application No. 60/839,001 filed Aug. 21, 2006 and U.S. Provisional Application No. 60/843,171 filed Sep. 8, 2006, each of which is hereby incorporated by reference in its entirety.

### STATEMENT REGARDING GOVERNMENT SPONSORED RESEARCH

Aspects of this work were supported by grant no. CT 503323315 from the National Science Foundation. The United States Government has certain rights in the subject matter.

### TECHNICAL FIELD

The present invention relates bioassays. More specifically, the present invention relates to bioassays using biopolymer-dye complexes.

### BACKGROUND

Efforts have been made to create ever more simple, rapid, reliable and sensitive bioassays. Such assays may be used for a myriad of functions including screening of biological molecules, chemical molecules, whole cells, organisms, and pathogens for use in drug discovery, bioagent detection and diagnostics.

Bioassays frequently use fluorescence-based detection. The availability of commercial instrumentation able to read fluorescence enhances the attractiveness of new assays employing fluorescence as a switch or signal.

Fluorescent dyes are widely used in applications which require highly sensitive detection. Changes in the amount of fluorescence enable researchers to determine the presence, quantity or location of molecules of interest. The quenching and energy transfer properties of fluorescent dyes also render the dyes useful for a variety of methods permitting investigators to monitor the in vivo and in vitro interactions between molecules. Among the fluorescent dyes which have been developed for use in biological, biochemical or chemical applications are a number of cyanine dyes.

Cyanine dyes have long been of interest to the scientific community due to their unique optical properties. Cyanine dyes are generally characterized by the presence of a pair of nitrogen-containing heterocycles connected by a polymethine bridge over which bond resonance occurs. Most cyanine dyes exhibit high visible absorbance and reasonable resistance to photodegradation.

Certain cyanine dyes form molecular aggregates that are either J-type or H-type depending upon their chemical structure and media. H aggregates have a blue-shifted absorption band, relative to the absorption wavelength of the monomeric dye. J-aggregates, on the other hand, have a narrower, red-shifted absorption band, compared to the monomer and also show a sharp, intense fluorescence emission compared to the monomer or other aggregates. The sharp emission spectra of J-aggregated cyanines and the structural diversity of the cyanines have made them attractive candidates for fluorescence-based sensing applications. However, although J-aggregation has been observed in a variety of environments, it is still unclear which factors tip the balance in favor of J-aggregation for structurally similar families of cyanines. For example, it has been found that while certain cyanines form J-aggregates when adsorbed onto clay nanoparticles, seemingly structurally similar cyanines may exhibit H-aggregation or dimerization when bound to the same type of clay nanoparticles. Additionally, in many cases the potential utility of the cyanine dyes is impaired by the low quantum efficiency for most J-aggregate fluorescence.

There therefore exists a need for the identification of molecules that can reliably form J-aggregates as well as for the development of rapid, simple, robust and sensitive assays which can take advantage of the particular properties of cyanine dyes.

### BRIEF DESCRIPTION OF THE DRAWINGS

FIG. 1 is a schematic representation of a scaffold destruction/formation assay in which a change in absorption spectra and/or fluorescence occurs as a consequence of supramolecular complex formation between a dye and a biopolymer.

FIG. 2 is a graph of the change in (a) absorption and (b) fluorescence (excitiation at 460 nm) in the titration of 0 to 30×10⁻⁵ M carboxymethylamylose (CMA) to 1×10⁻⁵M of cyanine dye of Formula I in a 20% methanol-water mixture.

FIG. 3 is a schematic of a scaffold destruction assay in which a helicase destroys a DNA:cyanine dye complex leading to fluorescence attenuation.

FIG. 4 is a schematic of a scaffold destruction assay in which a helicase unwinds a DNA:cyanine dye complex prior to peptide bond hydrolysis, decreasing the intensity of the fluorescence emission.

FIG. 5 is a schematic of a scaffold disruption assay in which a cyanine:DNA complex is treated with helicase, unwinding the DNA and decreasing the fluorescence of the sample.

FIG. 6 is a schematic of a lateral flow assay in which a dye/scaffold complex is introduced into a strip or channel in which there are anchored reagents to intercept the intact dye/scaffold complex as well as “released” dye.

FIG. 7 is a schematic for a sandwich based assay using fluorescence/absorption based sensing technology.

FIGS. 8a-d show schematicsschematics for positive (FIG. 8a-c) and negative (FIG. 8d) reactionsreactions for a sandwich based assay using fluorescence/absorption based sensing technology.

FIG. 9 is a schematic for a Lateral Flow Enzymatic reaction (LFER) assay using fluorescence/absorption based sensing technology.

FIG. 10 is a schematic for a variation of a Lateral Flow Enzymatic reaction (LFER) assay using fluorescence/absorption based sensing technology.

FIG. 11 is a schematic for a Later Flow Enzymatic/Immuno reaction (LFEIR) assay using fluorescence/absorption based sensing technology.

FIG. 12 is a schematic of a scaffold formation assay using cyanine tethering.

FIG. 13 is a schematic of a scaffold formation assay using encapsulation.

FIG. 14 is a schematic of a scaffold formation assay in which fluorescence is increased by the formation of cyanine conjugated oligomers.

FIG. 15 is a schematic of an assay devised to measure the activity of a hydrolytic enzyme cleaving a tethered dye-biopolymer conjugate.

FIG. 16 is the schematic for a Lateral Flow Immunoassay (LFIA) assay using fluorescence/absorption based sensing technology.

FIG. 17 is a schematic for a Lateral Flow Displacement Assay (LFDA) using fluorescence/absorption based sensing technology.

FIG. 18 is a schematic for a lateral Flow without PCR (LFWP) assay using fluorescence/absorption based sensing technology.

FIG. 19 is a chart displaying the time course of a scaffold disruption assay created by incubating amylase with a cyanine dye:CMA conjugate.

FIG. 20 is a chart depicting the effect of an increase in amylase concentration on the fluorescence emission of a cyanine dye:CMA conjugate.

FIG. 21 is a chart depicting the change in fluorescence emission immediately after combining the indicated amounts of carboxymethyl cellulose and 10 μM of cyanine dye.

FIG. 22 is a chart depicting the change in fluorescence emission immediately after combining the indicated amounts of cyanine dye and 10, 20 or 40 μM of carboxymethyl cellulose respectively.

FIG. 23 is a chart depicting the change in fluorescence emission sixty minutes after the creation of a reaction mixture created by adding increasing amounts of hyaluronic acid to 20 μM of cyanine dye.

FIG. 24 is a chart depicting the change in fluorescence emission sixty minutes after the creation of a reaction mixture created by adding increasing amounts of hyaluronic acid to 20 μM of cyanine dye.

FIG. 25 is a chart depicting the change in fluorescence emission sixty minutes after combining 1.0 μg of hyaluronic acid with the indicated amounts of cyanine dye.

FIG. 26 is a chart depicting the change in fluorescence emission sixty minutes after adding a reaction mixture combining 11.3 U of hyaluronidase with 8 μg of hyaluronic acid and incubated for the times indicated to wells containing 20 μM of cyanine dye of Formula I.

FIG. 27 is a chart depicting the change in fluorescence emission sixty minutes after reaction mixtures containing the indicated amounts of hyaluronidase and 10 μg of hyaluronic acid were added to 20 μM of cyanine dye of Formula I.

FIG. 28 is a chart depicting the change in fluorescence emission sixty minutes after reaction mixtures containing the indicated amounts of hyaluronic acid and 1 U of hyaluronidase was added to wells containing 20 μM of cyanine dye of Formula I.

### DETAILED DESCRIPTION

The present invention provides novel assays for use in drug discovery, bioagent detection and medical diagnostics. The present invention additionally provides assays based on cooperative self-assembly of complexes between biopolymers and fluorescent dyes. Such assays may be based on the formation, disruption and destruction of the complexes.

The unique optical properties of cyanine dyes have long held the interest of the scientific community. Cyanine dyes form several different types of aggregates characterized by spectral shifts from the emissions of the monomer. These spectral shifts may be formed depending on the molecular environment, concentration and physical state of the dye. Of the various aggregate types observered, the J-aggregates, characterized by very narrow intense absorption and narrow, only slightly red-shifted fluorescence relative to the monomer dye, have been the focus of most interest. Although J-aggregation has been observed in a variety of environments, it has previously been unclear which factors tip the balance in favor of J-aggregation for structurally similar families of cyanines.

Studies have shown that a series of polyelectrolytes with a variable number of cationic polymer repeat units constructed with cyanine chromophores pendant, but not conjugated, on a poly-L-lysine backbone, exhibit characteristic J-aggregate absorption and fluorescence in aqueous solution and when adsorbed onto anionic supports. For fluorescence of the polymers in solution, J-aggregate fluorescence became prominent for polymers having at least 33 cationic polymer repeat units. The fluorescence of the polymer is subject to very strong “super-quenching” by oppositely charged electron acceptors and energy-transfer quenchers that equals or exceeds that observed for conjugated polyelectrolytes by the same kind of quenchers. (Whitten et al., Pure Appl. Chem., Vol. 78, No. 12, pp. 2313-2323 (2006), incorporated by reference in its entirety.) Any cyanine dye capable of exhibiting J-aggregate fluorescence and absorption spectra when activated may be used in the assays of the present invention. In some embodiments, the ideal cyanine dye candidates would be ones where in aqueous solution there is no J-aggregate or only weak J-aggregate fluorescence but once complexed with a biomolecule the cyanine converts to J-aggregate and exhibits strong J-aggregate fluorescence. In exemplary embodiments, a cyanine dye of formula I, below, is used. This cyanine dye exhibits moderate water solubility and is nearly non-fluorescent in aqueous media.

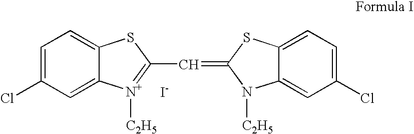

According to various embodiments of the present invention, certain cyanine dyes and related chromophores, including but not limited to, cyanine dyes of Formula I, can form stable, tight complexes with chiral polymeric molecules in aqueous solutions resulting in the formation of intensely fluorescent J-aggregates. This molecular aggregation occurs via cooperative self assembly in which both the dye and the polymer undergo conformational changes to adopt a supramolecular helical structure. In some embodiments, a charged cyanine dye may associate with an uncharged or oppositely charge biopolymer. It may be desirable for chirality to be taken into account, for example, when detection methods such as induced circular dichroism are used.

Geometrics of nature have been the subject of intense interest to chemists, biologists, physicists and mathematicians. Molecular and supramolecular aggregates assemble into interesting geometries such as helices through non-convalent interactions. Chem-bio-helices play diverse roles in a variety of scientific disciplines including chemistry, biology and physics. These helices illustrate an ordered, 3-dimensional geometry present in natural and synthetic molecular entities such as deoxyribonucleic acid, ribonucleic acid and collagen. In addition, proteins and carbohydrates with helical structures participate in key biological processes such as signal transduction, cartilage formation, joint lubrication, and host-pathogen interactions. Helical structures are also prominent in chemistry, for example, the carbon nanotube.

Evidence supporting the cooperative self-assembly of cyanine dyes has been obtained from atomic force microscopy and circular dichroism spectroscopy. See, e.g. Kim, O.-K.; Je, J.; Jernigan, G.; Buckley, L.; Whitten, D. J. Amer. Chem. Soc. 128, 510-516 (2006), which is hereby incorporated by reference.

The assays formed by the methods herein may be used to detect both linear and helical biopolymers as well as non-polymeric small molecular weight analytes at a variety of concentrations, including, but not limited to, picomolar and femtomolar concentrations, and may be adapted to the format best suited for the detection of the analyte in question. In some embodiments, the assays herein use cyanine dyes to form chem-bio-helices with chem-biopolymers such as nucleic acids, proteins including helical and partially helical proteins, carbohydrates, linear and helical lipids, foldamers, linear chemical polymers and chemical helicates. (H. Gorner, A. K. Chibisov, T. D. Slavnova, J. Phys. Chem. B110:3917-3923 (2006); A. S. Tatikolov, I. G. Panova, High Energy Chem. 39: 232-236, (2005); and I. G. Panova, A. S. Tatikolov., Doklady Biol Sci. 402:183-185 (2005), each of which is incorporated by reference herein in its entirety), linear polymers (O-K. Kim, J. Je, J. W. Baldwin, S. Kooi, P. E. Pehrsson, L. J. Buckley., J. Am. Chem. Soc. 125L4426-4427 (2003); O-K Kim, J. Je, G. Jernigan, L. Buckley, D. Whitten. J. Am. Chem. Soc. 128:510-516 (2006); and D. Whitten, Ok>k Kim, K. E. Achyuthan, “Cooperative Self-Assembly of Cyanines on Carboxymethylamylose and other Anionic Scaffolds” IUPAC Mtg., Kyoto, Japan, April, 2006; each of which is incorporated by reference herein in its entirety) and carbohydrates. Exemplary biopolymers include, but are not limited to, linear polysaccharides such as cellulose, including carboxymethylamylose (CMA) (O-K Kim, J. Je, G. Jernigan, L. Buckley, D. Whitten., J. Amer. Chem. Soc. 128, 510-516 (2006), incorporated by reference herein in its entirety) and carboxymethylcellulose (CMC), and glycosaminoglycans including hyaluronic acid (G. Weindl, M. Schaller, M. Schafer-Korting, G. C. Korting. Skin Pharmacol. Physiol. 17:207-213 (2004); T. Sagawa, H. Tobata, H. Ihara. Chem. Comm. 18:2090-2091 (2004); S. Arnott, A. K. Mitra. S. Raghunathan. J. Mol. Biol. 169:861-872 (1983), each of which is incorporated by reference herein in its entirety); DNA; RNA; and proteins or structures composed of proteins such as albumin (I. G. Panova, A. S. Tatikolov., Doklady Biol. Sci. 402:183-185 (2005), incorporated by reference herein in its entirety), Bacillus collagen-like antigen (S. Rety, S. Salamitou, I. Garcia-Verdugo, D. J. S. Holmes, F. Le Hegarat, R. Chaby, A. Lewit-Bentley, J. Bio. Chem. 280:43073-43078 (2005), incorporated by reference herein in its entirety), cell membranes (M. Reers, T. W. Smith, L. B. Chen, Biochemistry 30:4480-4486 (1991), incorporated by reference herein in its entirety), phospholipids bilayers K. L. Vedvik, H. C. Eliason, R. L. Hoffman, J. R. Gibson, K. R. Kupcho, R. L. Somberg, K. W. Vogel, Assay Drug Dev. Technol. 2:193-203 (2004), incorporated by reference herein in its entirety), liposomes (H. F. Gilbert, “Basic Concepts in Biochemistry”, McGraw-Hill, Inc., New York, pp. 81-108 (1992), incorporated by reference herein in its entirety), reverse micelles S. M. Andrade, S. M. B. Costa, Chem. Eur. J. 12:1046-1057 (2006), incorporated by reference herein in its entirety), gelatin (H. Gorner, A. K. Chibisov, T. D. Slavnova, J. Phys. Chem. B110:3917-3923 (2006), incorporated by reference herein in its entirety), and patho-physiologically vital proteins including, but not limited to, potassium channel displaying extensive helices (D. A. Doyle, J. M. Cabral, R. A. Pfuetzner, A. Kuo, J. M. Gulbis, S. L. Cohen, B. T. Chait, R. MacKinnon., Science 280:69-77 (1998), incorporated by reference herein in its entirety) or the Streptococcal phage-encoded virulence factor (N. L. Smity, E. J. Taylor, A-M Lindsay, S. J. Charnock, J. P. Turkenburg, E. J. Dodson, G. J. Davies, G. W. Black, Proc. Natl. Acad. Sci. USA 102:17652-17657 (2005), incorporated by reference herein in its entirety). The creation, destruction, and/or disruption of such biopolymer:cyanine dye helices may be used to determine the presence, absence and amount of the biopolymer or enzyme of interest.

Changes associated with the formation of J-aggregation and changes in fluorescence and emission spectra of cyanine dye complexes may be read by means well known to those of skill in the art. There are a number of commercially available UV and fluorescence readers capable of viewing and quantifying such changes. In some embodiments, ratiometric analyses of the changes will be used. Such analyses may increase the specificity of the assay.

Assays of the present invention utilizing both the absorption and emissive properties for the light and spectral changes associated with the formation of J-aggregates of cyanine dyes may take a variety of forms. Such assays may be of any type generally used by those skilled in the art including, but not limited to, direct assays; indirect assays such as sandwich assays; competitive assays; non-competitive assays; vertical (columnar) format assays; and lateral flow assays such as, but not limited to, lateral flow enzymatic reaction assays, lateral flow immunoassays, displacement assays, hybrid lateral flow enzymatic/immunoreaction assays, and lateral flow displacement assays. In some embodiments, the assays of the present invention may be used in high throughput screenings.

In some embodiments, assays may be conducted using solid phase supports. Such solid phase supports may include any solid phase or semi-porous surface known to those of skill in the art including, but not limited to, glass, polystyrene, polypropylene, nitrocellulose, latex, cellulose, agarose, clay, silica, dextran or other materials. Suitable forms of the solid phase supports include beads, microparticles, tubes, fabrics, plates, latex particles, magnetic particles, paper, dipsticks, nano-particles, coupons, or tickets, formed from or coated with these materials as well as alternative flow-based formats such as channels and multiplexed assays on patterned substrates and the like (D. M. Olive., Expert Rev. Proteomics 1:327-341 (2004); W. Miaomiao, G. L. Silva, B. A. Armitage, J. Am. Chem. Soc. 122:9977-9986 (2000); K. M. Sovenhazy, J. A. Bordelon, J. T. Petty, Nucleic Acids Res. 31;2561-2569 (2003); Robert M. Jones, Liangde Lu, Roger Helgeson, Troy S. Bergstedt, Duncan W. McBranch, and David G. Whitten, Proc. Natl. Acad. Sci. USA 98:14769-14772 (2001); R. Jones, T. Bergstedt, C. Buscher, D. McBranch, D. Whitten, Langmuir 17:2568 (2001) and L. Lu, R. M. Jones, D. McBranch, D. Whitten, Langmuir 18:7706-7713 (2002), each of which is incorporated by reference herein in its entirety). In some embodiments, the assay may test for a single analyte. In other embodiments, the assay may test for multiple analytes including 2, 3, 4, or more analytes In some embodiments, as many as 100 or more analytes may be used. For example, those of skill in the art will be familiar with assays available from Luminex Corporation (Austin, Tex.) that include dye-labeled microspheres capable of testing 100 different analytes within a single sample. A similar system could be used incorporating the systems and methods described herein.

In some embodiments, the analytes may be labeled by means other than or in addition to bonding with a cyanine dye. Such labeling may include any means of generating a detectable signal. Illustrative labeling includes, but is not limited to, chromogens; catalysts, both enzymatic and non-enzymatic; molecules having an enzymatically labile bond which upon enzymatic cleavage provides a compound that can be detected either directly or indirectly; labeling with magnetic particles and isotopic labeling. In other embodiments, FRET labeled reagents that either sensitize or quench cyanine aggregate fluorescence may be utilized. In still other embodiments, the analytes may be unlabeled removing the need to chemically modify either the substrate or the enzyme. The ability of the assays of the present invention to use substrates in their natural conformational states provides a greater degree of confidence regarding the validity of the data to mimic in vivo conditions.

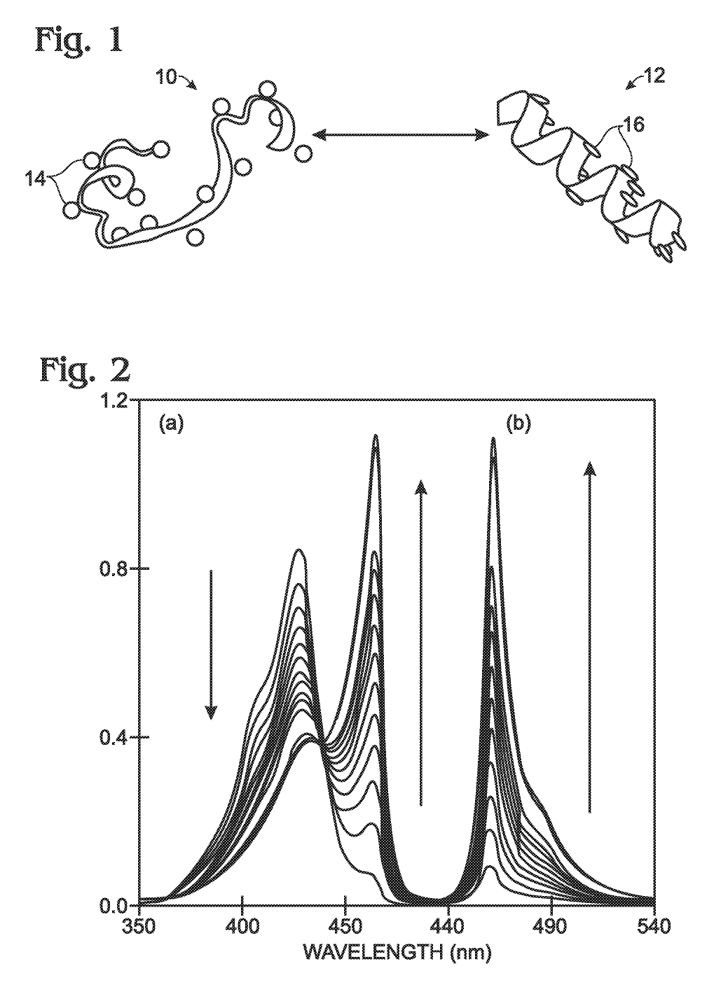

The assays of the present invention may function in a biosynthetic or metabolic fashion increasing or decreasing fluorescence as well as shifting the absorption spectrum emitted by the cyanine dyes. In some embodiments, the assays of the present invention may function in a biosynthetic fashion such as scaffold formation of a cyanine dye:biopolymer helix. In other embodiments, the assays may be based on metabolic processes such as scaffold destruction or disruption of a cyanine dye:biopolymer helix. A schematic representation of a scaffold formation/scaffold destruction assay based on complex formation between a biopolymer and a dye is shown in FIG. 1, in which a change in absorption and/or fluorescence occurs as a consequence of supramolecular complex formation/destruction. In FIG. 1, a random coil polymer 10 and a supra-molecular bio-helix 12 are shown. Random coil polymer 10 includes cyanine dye 14 and is beginning to assemble on scaffold. In this conformation, the “light” (i.e. detectable fluorescence or absorption spectrum is considered to be “off.” Supra-molecular bio-helix 12 shows formed cyanine J-aggregates 16. In this confirmation, the “light” is considered to be “on.” The conversion of the random coil polymer on the left to the helical structure with the J-aggregated cyanine may be a cooperative process. J-aggregation may also occur on a pre-formed helical scaffolding and the “tightness” (or reversibility) of the J-aggregate-biopolymer complex may depend on the structure of the dye and the specific biopolymer. Change from one conformation to the other may be produced via the introduction of or exposure to scaffold forming or disrupting enzymes and/or triggers. In some embodiments, scaffold disruption assays may be created in which the disruption effects the distribution of the reagents in the assay, but the fluorescence signal and/or absorption spectrum does not change. In such embodiments, the presence and concentration of the analyte may be determined by the displacement of the signal rather than a change in intensity.

Assays of the present invention may indicate the presence or absence of an analyte as well as the amount of the analyte of interest based on changes in the fluorescence and/or absorption spectra or lack thereof emitted by a biopolymer:dye complex. For example, a biopolymer may form a chem-bio-helix with cyanine dye, resulting in a change in the fluorescence of the cyanine dye in comparison to the monomer and/or a shift in the absorption spectrum.

FIG. 2 is a graph of the change in (a) absorption and (b) fluorescence (excitation at 460 nm) in the titration of 0 to 30×10⁻⁵ M carboxymethylamylose (CMA) to 1×10⁻⁵M of cyanine dye of Formula I in a 20% methanol-water mixture. As shown in FIG. 2, the intensity of the fluorescence is an indication of the amount of the biopolymer present. The addition of an enzyme or a sample suspected of containing an enzyme that disrupts or degrades the biopolymer would alter the amount of fluorescence and/or alter the absorption spectrum of the cyanine dye:biopolymer mixture indicating the presence of an enzyme or identity of a particular biopolymer.

According to various embodiments, a wide variety of enzyme/biopolymer pairs may be used or detected by the assays of the present invention. Exemplary enzymes which may be used in or may be detected by the assays of the present invention may include, but are not limited to, amylases, hyaluronidases, DNases, RNases, helicases, gyrases, cellulases, lipases including phospholipases, various synthases (i.e. biosynthetic enzymes such as DNA polymerases) especially those that lead to biochemical synthesis of helices such as DNA, RNA, cellulose, amylose, or collagenases.

In one embodiment, cyanine dye may be combined with a carbohydrate such as, but not limited to, carboxymethylamylose (CMA). Such a combination results in the formation of a J-aggregate and thus a change of the absorption spectrum of the dye (FIG. 2). As shown in FIG. 2 this combination also results in a significant increase in the fluorescence of the J-aggregate. The addition of an enzyme that breaks down carbohydrates such as amylase would decrease the fluorescence and shift the absorption spectrum of the reaction mixture.

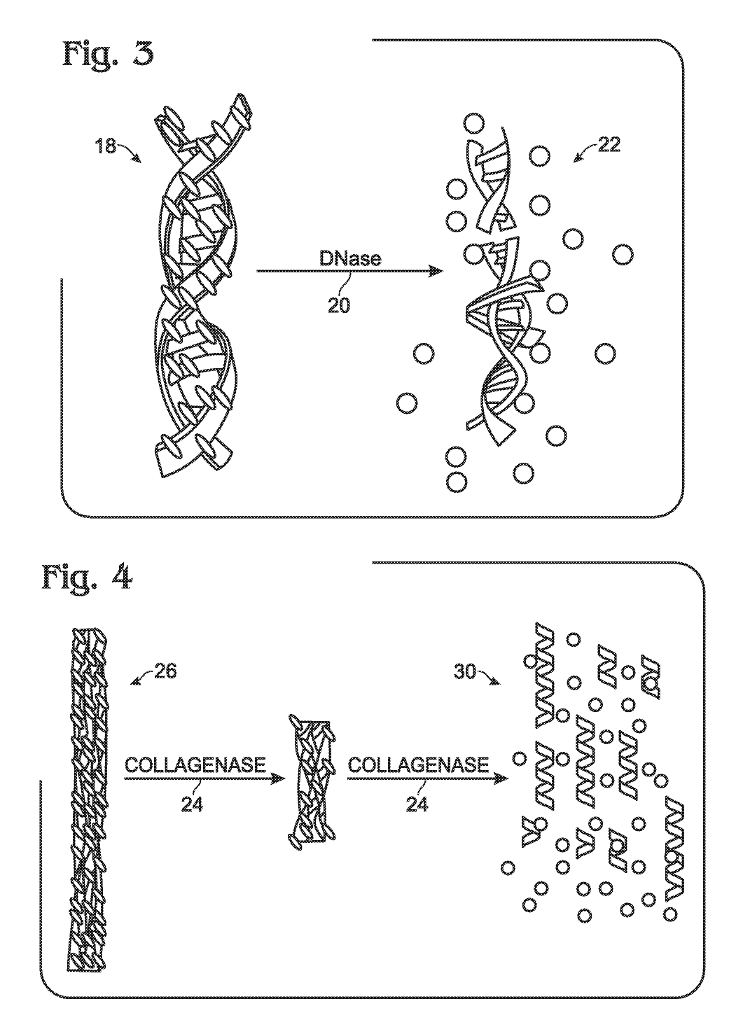

Scaffold destruction assays may also involve helicases. For example, in some embodiments cyanine dye may assemble on a DNA duplex. The addition of a sample containing a helicase may destroy the DNA scaffolding as shown in FIG. 3, where cyanine assembled on a DNA duplex is shown at 18 (fluorescence “on”) and cyanine dis-assembled due to DNA Scaffold Destruction via the introduction of DNase 20 is shown at 22 (fluorescence “off”). In this scaffold destruction assay, the helicase has destroyed the DNA:cyanine dye complex 18, leading to fluorescence attenuation. In other embodiments, as shown in FIG. 4, the addition of a protease such as collagenase 24 unwinds the triple helix 26 (fluorescence “on”) prior to peptide bond hydrolysis ((shown at 30, fluorescence “off”). Such assays are particularly useful in the detection of pathophysiologically vital proteins including, but not limited to, Streptococcal phage-encoded virulence factor as well as detection of viral diseases including, but not limited to, hepatitis C (HCV), herpes simplex virus (HSV) and human immunodeficiency virus (HIV) as well as genetic disorders such as xeroderma pigmentosum, Cockayne syndrome, or trichothiodystrophy, Bloom syndrome, Werner syndrome, Rothmund-Thomson syndrome. (N. Tuteja, R. Tuteja, Eur. J. Biochem. 271:1835-1848 (2004), incorporated by reference herein in its entirety). These assays may also be extended to pathophysiologically important carbohydrates such as hyaluronic acid that is implicated in osteoarthritis, liver fibrosis and liver cirrhosis. Similarly, lipases may be assayed using this technology for assessing risks associated with obesity or the onset of pancreatitis.

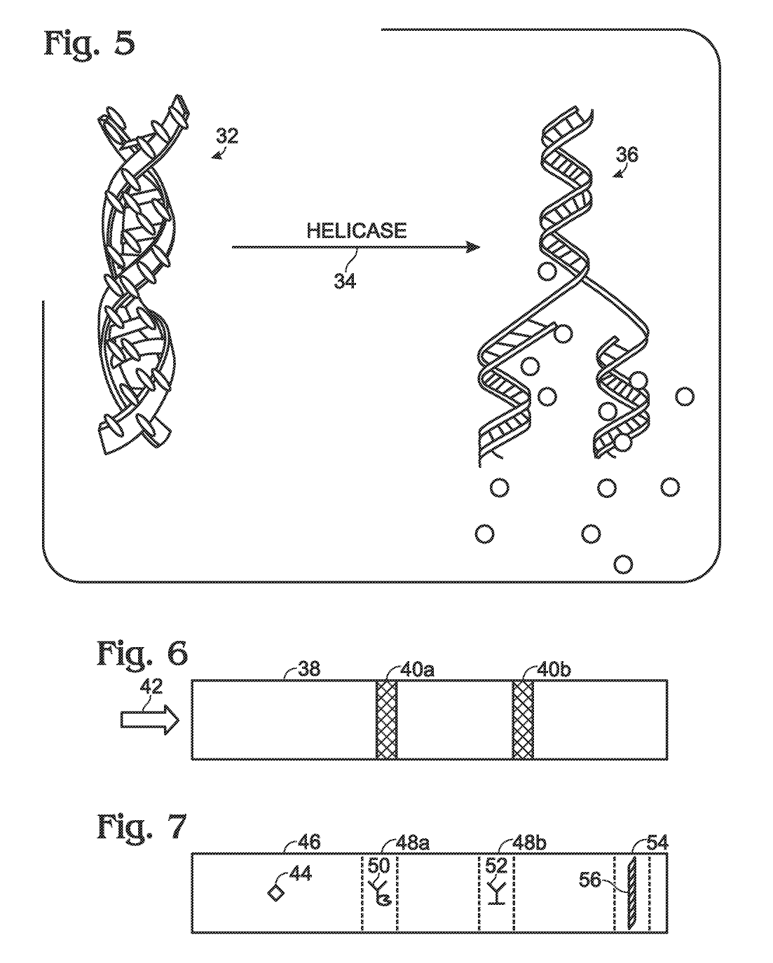

In another embodiment, instead of a scaffold destruction assay, the assay may be a scaffold disruption assay as shown in FIG. 5. For example, cyanine dye may assemble onto a DNA duplex 32. The addition of a helicase 34 may unwind the DNA, disrupting the scaffolding which leads to the cyanine becoming disassembled from the DNA duplex, as shown at 36 and decreases the fluorescence of the sample.

Assays of the present invention may be conducted by any means applicable, for example using a solid phase support. In some embodiments, one or more of the reagents may be pre-loaded on a solid phase support. For example, the solid phase support may contain cyanine dye, biopolymers, enzymes, cyanine dye: biopolymer complexes, or any combination thereof. In another embodiment, the solid phase support may contain one or more capture zones capable of binding cyanine dye, biopolymers, enzymes, biopolymer fragments, cyanine dye: biopolymers, analytes, or any combination thereof. Such capture zones may or may not contain additional labels capable of emitting a signal when a reaction is completed. In some embodiments, capture zones may include, but are not limited to, one or more reaction zones containing one or more bound antibody-enzyme conjugates or other recognition molecules bound to an enzyme that disrupts biopolymer:cyanine dye complexes, biopolymer:dye complexes, molecules specific for the biopolymer under investigation; one or more recognition zone containing molecules specific for biopolymers under investigation; one or more detection zones containing biopolymer:cyanine dye complexes; one or more test zones containing antibodies or other affinity ligands for intact biopolymer and/or biopolymer fragments, including but not limited to DNA strands, additional biopolymers capable of trapping cyanine dye; one or more control zones containing molecules capable of forming chem-bio helices with cyanine dye and/or producing the spectral changes associated with J-aggregate formation such as, but not limited to, clay nanoparticles, DNA or RNA duplexes, or biopolymers such as, but not limited to, linear polysaccharides such as cellulose, including carboxymethylamylose (CMA) and carboxymethylcellulose (CMC), and glycosaminoglycans; and proteins or structures composed of proteins such as albumin, Bacillus collagen-like antigen, cell membranes, phospholipids bilayers, liposomes, reverse micelles and gelatin, or any other appropriate molecules; as well as any other applicable capture zones. Antibodies, affinity ligands, biopolymers, cyanine dye or recognition molecules in the capture zones may be adsorbed, covalently linked, conjugated via ligand protein association or other non-covalent linkage, trapped or otherwise attached to or associated with the solid phase support. It is known to functionalize cyanine, accordingly it should be possible to biotinylate, cyanine. Biotinylated cyanine may be immobilized via streptavidin-biotin interactions. For example, in some embodiments, the molecules may be isolated in a capture zone using a size permeation filter. In another embodiment, an antibody may be adsorbed to the solid phase supports. In other embodiments, the antibodies, antibody-enzyme conjugates, affinity ligands, recognition molecules, or recognition molecule-enzyme conjugates may be mobile. Signals including, but not limited to, shifts in J-aggregate emissions, may be measured at some or all of the capture zones.

In one embodiment, each reagent may be introduced individually to the solid support. In a further embodiment, combinations of reagents may be introduced to the solid support, for example a sample may be combined with cyanine dye. Alternatively, a sample may be combined with a cyanine dye:biopolymer complex before being introduced to the solid support. FIG. 6 is a schematic of a lateral flow assay in which a dye/scaffold complex is introduced into a strip or channel 38 in which there are anchored reagents to intercept the intact dye/scaffold complex as well as “released” dye. For example, reagent located in region 40a may be an anchored antibody or other reagent that can capture the scaffold molecule and scaffold/dye complex. Alternatively or additionally, region 40a may include a size permeation filter or other suitable structure/reagents capable of sorting or otherwise distinguishing various components of the sample. Furthermore, reagent located in region 40b may be able to capture the dye and cause it to form a highly luminescent or colored aggregate. Arrows 42 indicate an exemplary flow direction. In another embodiment, a sample suspected of containing a biopolymer of interest may be combined with an enzyme that degrades the polymer and the combined mixture may be introduced to the solid support. In a further embodiment, a sample suspected of containing an enzyme of interest may be combined with a biopolymer degraded by that enzyme prior to introduction of the sample to the solid support. In additional embodiments, enzymes or biopolymers may be labeled prior to introduction to the solid phase support.

In one embodiment, the capture zone on a solid phase support may be a reaction zone. The reaction zone may contain antibodies or other recognition molecules specific for the analyte to be detected. As seen in FIG. 7, the sample containing the item of interest 44 may be introduced to the solid phase support 46 containing one or more capture zones 48a, 48b. As the analyte crosses the first reaction zone 48a, it binds to the mobile antibody or other recognition molecule conjugated to a disruptive enzyme 50. The solution then crosses a second reaction zone 48b containing bound second antibody or other recognition molecule 52 specific for the analyte of interest and binds the mobile analyte-antibody-enzyme conjugate. Free enzyme-antibody conjugate continues along the solid phase support until it reaches the detection zone 54 containing the biopolymer:cyanine dye complex 56. It should be appreciated that similar assays can be easily constructed in vertical flow (flow through) formats.

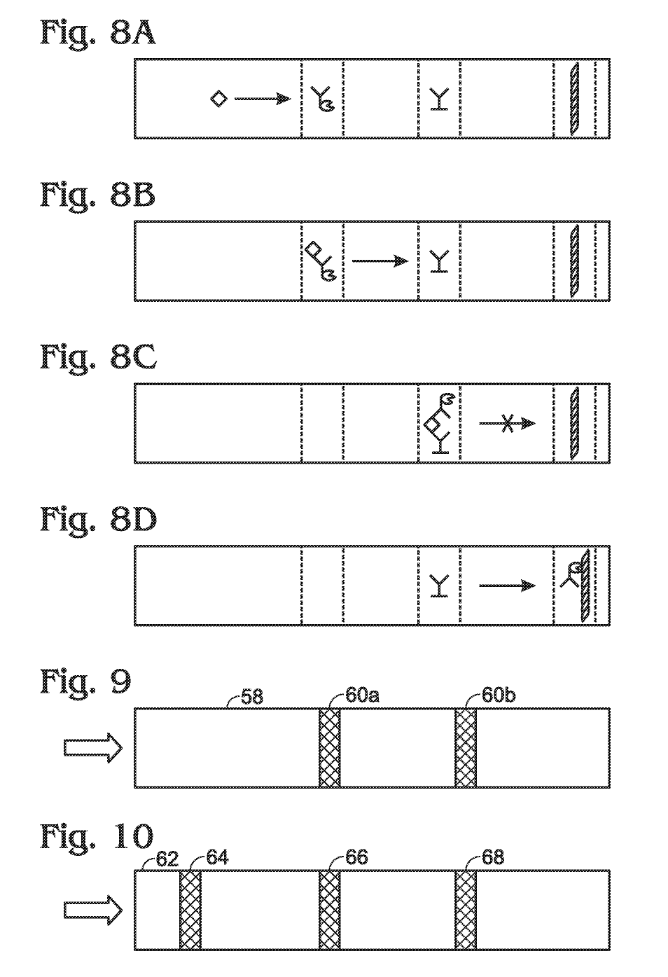

In various embodiments, sandwich-based assays such as that shown schematically in FIGS. 8a-d may be conducted using the presently described techniques. In the depicted embodiment, the analyte (if present) reacts with an antibody carrying a reagent capable of forming a complex with the cyanine and travels to the second zone where there is an anchored antibody capable of trapping the analyte-antibody complex. When the cyanine is wicked into the test zone, it forms a colored complex with the “sandwich”. Any untrapped dye travels to the control strip so that color may develop on the second strip or control (end strip) depending on the amount of analyte present. Minimal or the absence of a change in the emissions and absorption spectra in the detection zone indicates a positive result (FIGS. 8a-c), whereas complete or a strong change in the emissions and absorption spectra in the detection zone (FIG. 8d) would indicate the absence of the analyte of interest.

In another embodiment, the assay may be a lateral flow enzymatic reaction (LFER) assay. An exemplary embodiment of a LFER assay is shown in FIG. 9. In a LFER assay, a sample potentially containing the analyte of interest may be mixed with its suspected polymeric substrate. For example, a sample containing or suspected of containing amylase may be combined with carboxymethyl amylose or a sample containing or suspected of containing hyaluronidase may be combined with hyaluronic acid. After allowing the enzymatic reaction to take place under the appropriate conditions of time, temperature, and buffer solutions, the mixture may be introduced to a solid phase support 58 containing one or more capture zones 60a, 60b as shown in FIG. 9. The appropriate conditions of time, temperature and buffer solutions are readily established or well known to those of skill in the art. Capture zones may include one or more test zones which contain antibodies or other affinity ligands to the intact polymer to immobilize any residual intact polymer, and one or more control zones which may contain molecules capable of forming chem-bio helices with cyanine dye and/or producing the spectral changes associated with J-aggregate formation, or any other capture zone appropriate for the assay. In some embodiments, the control zone may contain clay nanoparticles, DNA or RNA duplexes, or other biopolymers such as, but not limited to, linear polysaccharides such as cellulose, including carboxymethylamylose (CMA) and carboxymethylcellulose (CMC), and glycosaminoglycans; and proteins or structures composed of proteins such as albumin, Bacillus collagen-like antigen, cell membranes, phospholipids bilayers, liposomes, reverse micelles and gelatin, or any other appropriate molecules. In some embodiments, the control zone may contain the biopolymer used to test for the analyte of interest. Accordingly, in one exemplary assay, the analyte and polymeric substrate which has been incubated and mixed with the dye, or which contains an “on-board” dye may be introduced to a strip or channel 58 containing a test zone 60a, configured to trap the intact polymer or polymer-dye complex and a control zone 60b, which may contain polymer clay, or some other reagent templating Dye aggregate.

In one embodiment, once the enzyme/polymer reaction mixture has been introduced to the solid phase support, cyanine dye may be added and allowed to travel along the solid phase support, binding to the biopolymer in the capture zones. The difference in the light absorption and emission intensities between the capture zones is an index of the enzymatic activity in the sample. In some embodiments, the difference in the light absorption and emission intensities may be used to determine the presence or absence of the analyte of interest. In other embodiments, the difference in the light emission intensities may be used to determine the amount present of the analyte of interest.

In one embodiment, the LFER assay may additionally include a capture zone such as a reaction zone containing antibodies or other affinity ligands to biopolymer:cyanine dye complexes. An embodiment of this assay is shown in FIG. 10. As shown in FIG. 10, a solution containing the biopolymer:cyanine dye complex may be introduced to a solid phase support 62, binding to the reaction zone. The binding of the biopolymer:cyanine dye complex may cause the zone to produce the spectral changes associated with J-aggregate formation. The sample potentially containing the enzyme of interest may then be introduced to the solid phase support. If the sample contains the enzyme of interest, it will digest the captured biopolymer:cyanine dye complex in the reaction zone, decreasing the fluorescence in the reaction zone. In one embodiment, the LFER assay may include a reaction zone 64 in conjunction with a test zone 66 and control capture zone 68. The reaction zone 64 may include, for example, an imprinted biopolymer-dye complex. The test zone 66 may include, for example, an anchored polymer, clay, or other reagent capable of trapping the dye. The control zone 68, may include, for example, polymer clay, or other reagent templating Dye aggregate.

In some embodiments, the LFER assay may include multiple test zones that contain antibodies or recognition elements to bind either or both the biopolymer fragments and the liberated cyanine dye. In one embodiment, the test zone may contain a second biopolymer that serves to capture a portion of the liberated cyanine. The remaining liberated cyanine may then bind to other cyanine binding entities in the control zone. A positive test result may be indicated by the formation of J-aggregates in the test and control zones. In a further embodiment, the test and control zones may contain recognition elements that bind to the biopolymer fragments. Such elements may contain one or more labels that are activated when the biopolymer fragments are bound. The activation of such markers in the test and control zone would indicate a positive test result. In additional embodiments, the test zone and/or the control zone may contain antibodies or affinity ligands to either biopolymers or cyanine dye. In yet another embodiment, the LFER assay may contain a reaction zone and either a test zone or a control zone.

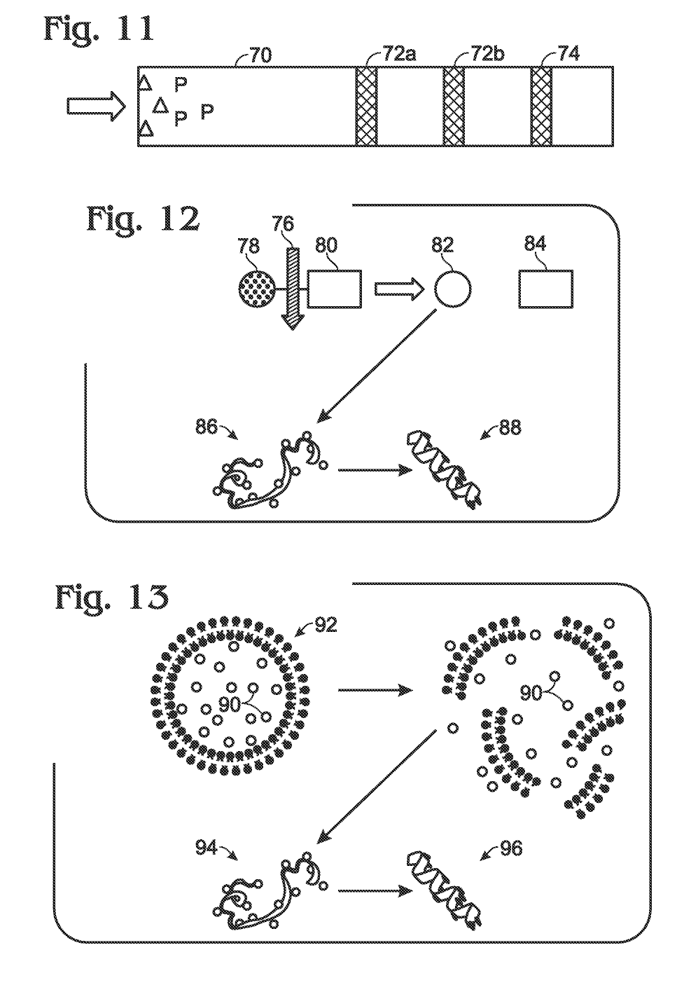

Assays may additionally comprise hybrid lateral flow enzymatic/immunoreaction (LFEIR) assays. An example of such an assay is shown in FIG. 11. In a LFEIR assay, a biopolymer is combined with a test sample potentially containing an enzyme of interest. In some embodiments, the combination of the biopolymer with the sample will result in complete digestion of the biopolymer. In other embodiments, there may be varying amounts of intact polymer remaining. After allowing the enzymatic reaction to take place under the appropriate conditions of time, temperature, and buffer solutions as are readily established or known to those of skill in the art, the mixture is introduced to a solid phase support containing one or more capture zones 72, 74. Capture zones may include one or more test zones (72a, 72b) which contain immobilized antibodies or other affinity ligands specific for the intact polymer or polymer fragments and one or more control zones which may contain molecules capable of forming chem-bio helices with cyanine dye and/or producing the spectral changes associated with J-aggregate formation, or any other capture zone appropriate for the assay. In some embodiments, the control zone 74 may contain clay nanoparticles, DNA or RNA duplexes, or other biopolymers such as, linear polysaccharides such as cellulose, including carboxymethylamylose (CMA) and carboxymethylcellulose (CMC), and glycosaminoglycans; DNA; RNA; and proteins or structures composed of proteins such as albumin, Bacillus collagen-like antigen, cell membranes, phospholipids bilayers, liposomes, reverse micelles, gelatin, and pathophysiologically vital proteins including, but not limited to, potassium channel displaying extensive helices or the Streptococcal phage-encoded virulence factor.

In one embodiment, as shown in FIG. 11, the LFEIR assay may contain capture zones for the intact polymer, polymer fragments and a control respectively. After the introduction of the reaction mixture to the solid phase support, any intact polymer may be isolated at one capture zone while any polymer fragments may be isolated at a second capture zone. For example, test zone 72a may contain immobilized antibodies to the biopolymer, while test zone 72b may contain immobilized antibodies to polymer fragments. Cyanine dye may then be introduced to the solid phase support and allowed to bind to the polymer, polymer fragments and control. Light absorption and emission may then be measured at each of the capture zones. If the test sample contains the analyte of interest, at least the capture zone containing the polymer fragments and the control will produce the absorption and emission spectra characteristic of J-aggregates. If the test sample does not contain the analyte of interest, neither the capture zone containing affinity ligands for the intact biopolymer nor the capture zone containing affinity ligands for the biopolymer fragments will emit the characteristic absorption and emission spectra.

In some embodiments, the assays may be based on biosynthetic processes or scaffold formation. In scaffold formation assays, cyanine dyes are prevented from forming cyanine dye:biopolymer complexes that create J-aggregates. The addition of a compound that allows the cyanine dye:biopolymer complexes to form J-aggregates, creating a signal that can be measured. Such signals may reveal the presence and quantity of analytes of interest.

For example, the cyanine may be prevented from forming chem-bio-helices with a product by tethering the cyanine via covalent bonds to a suitable substrate as shown in FIG. 12. The tethered cyanine dye may be combined with a random coil polymer 86 and a hydrolytic enzyme 76. As shown in FIG. 12, the enzyme 76 cleaves the tethered cyanine 78 from substrate 80 producing free cyanine 82 and product 84. Free cyanine is then able to associate with the random-coil CMA at 86 to produce the cyanine/CMA superhelix shown at 88. Once liberated, the cyanine would form helices with the polymer, resulting in fluorescence or a “turn on” assay. The use of a non-signaling substrate which yields a signal upon hydrolysis has been used in testing for a variety of diseases including, but not limited to, tests for influenza viruses. (A. Liav, J. A. Hansjergen, K. E. Achyuthan, C. D. Shimasaki, Carbohydrate Res. 317: 198-203 (1999); K. E. Achyuthan, L. Pence, J. Applemann, C. D. Shimasaki., Kuminescence 18:131-139 (2003); M. S. Hamilton, D. M. Abel, Y. J. Ballam, M. K. Otto, A. F. Nickell, L. M. Pence, J. R. Appleman, C. D. Shimasaki, K. E. Achyuthan, J. Clin. Microbiol. 40: 2331-2334 (2002); K. E. Achyuthan, L. M. Pwence, D. R. Mantell, P. E. Nangeroni, D. M. Mauchan, W. M. Aitken, J. R. Appleman, C. D. Shimasaki. Luminescence 18: 79-89 (2003), each of which is incorporated by reference herein in its entirety).

Appropriate tethering compounds include any compound that forms a bond with cyanine that may be cleaved by hydrolytic enzymes such as, but not limited to, lipases, glycosidases, phosphatases, sialidases, caspases, influenza viral neuraminidases, proteases or peptidases. Tethering compounds may include, but are not limited to, lipases, proteins, DEVD, glycoside, sialic acid, or 4,7-Di-O-Me-N-Acetyl-Neuraminic Acid. Such compounds may form tethered substrates such as DEVD-cyanine, protein-cyanine, glycoside-O-cyanine, sialic acid-o-cyanine and 4,7-Di-O-Me-N-Acetyl-Neuraminic Acid-O-cyanine

In some embodiments, the tethered biopolymer-dye complex, and enzyme (analyte) may be pre-loaded on a solid phase surface. In another embodiment, the tethered biopolymer-dye complex may be pre-loaded on a solid phase surface. In a further embodiment, the analyte may be preloaded on a solid phase surface.

In another embodiment, the cyanine dye 90 may be encapsulated as shown at 92 in FIG. 13. Sequestration of the cyanine may be accomplished by using any encapsulation techniques known to those of skill in the art including, but not limited to, hydrogels, dendrites, nanoparticles, liposomes, phospholipid vesicles, erythrocyte ghosts, Inside Out Vesicles (IOVs), polyelectrolyte microshell carriers and molecular encapsulation. (W. S. W. Shalaby, G. E. Peck, K. Park, J. Control Release 16:355-364 (1991), incorporated by reference herein in its entirety), dendrites (D. Seebach, G. Herrmann, U. Lengweiler, B. Bachmann, Angew. Chem. 35:2795-2797 (1996), incorporated by reference in its entirety), nanoparticles (K. Kim, M. Lee, H. Park, J-H Kim, S. Kim, H. Chung, K. Choi, I-S. Kim, B. L. Seong, I. C. Kwon, J. Am. Chem. Soc. 128: 3490-3491 (2006), incorporated by reference in its entirety), liposomes (U. Raviv, D. J. Needleman, Y. Li, H. P. Miller, L. Wilson, C. R. Safinya, Proc. Natl. Acad. Sci. USA 102:11167-11172 (2005), incorporated by reference in its entirety), phospholipid vesicles (D. Fugman, K. Shirai, R. Jackson, J. Johnson, Biochim. Biophys. Acta 795:191-195 (1984); and R. Zeineldin, M. E. Piyasena, T. S. Bergstedt, L. A. Sklar, D. Whitten, G. P. Lopen, Cytomety 69A:335-341 (2006), each of which is incorporated by reference in its entirety), erythrocyte ghosts (J. M. Dugan, C. A. Dise, D. B. Goodman, Biochim. Biophys. Acta 816: 93-101 (1985), incorporated by reference in its entirety), Inside Out Vesicles (IOVs) (T. Kondo, G. L. Dale, E. Beutler, Proc. Natl. Acad. Sci. USA 99:4837-4841 (2002), incorporated by reference in its entirety), polyelectrolyte microshell carriers (M. J. McShane, J. Q. Brown, K. B. Guice, Y. M. Lvov, J. Nanosci. Nanotech. 2:411-416 (2002), incorporated by reference in its entirety) and molecular encapsulation (M. M. Conn, J. Rebek, Chem. Rev. 97:1647-1668 (1997), J. L. Atwood, et al., Proc. Natl. Acad. Sci. USA 99:4837-4841 (2002), and N. Sorde, G. Das, S. Matile, Proc. Natl. Acad. Sci. USA 100: 11964-11969 (2003), each of which is incorporated by reference herein in its entirety. In some embodiments, these capsules may contain recognition/reporter elements that when reacted upon by enzymes or other trigger molecules such as catalytic antibodies, detergents, or lipases to release the trapped cyanine (E. Keinan (ed). “Catalytic Antibodies” Wiley VCH, Weinheim, Germany (2004), incorporated by reference herein in its entirety).

For example, cyanine may be incorporated in vesicles composed of a phospholipid such as 1,2-dipalmitoyl-sn-glycero-3-phosphocholine (DPPC). Cyanine dye will assemble on the bilayer walls of the vesicles composed of DPPC and result in fluorescence. Disruption of the vesicle by the action of an appropriate phospholipase or a detergent such as Triton X-100 will result in disaggregation and optical activity being switched off.

In another example, cyanine dyes may be incorporated inside hydrogels that may be proteolytically degraded resulting in the liberation of the “free” cyanine which then assembles upon a suitable chem-bio-polymer resulting in strong fluorescence emission signaling the proteolytic event. An example of a patho-physiologically important protease family is the matrix metalloproteinases (MMPs) such as collagenases, stromelysins, matrilysins and gelatinases. The MMPs play an important role in tissue remodeling associated with various physiological and pathological processes such as morphogenesis, angiogenesis, tissue repair, cirrhosis, arthritis and metastasis. MMP-2 and MMP-9 are thought to be important in metastasis. MMP-1 is thought to be important in rheumatoid and osteo-arthritis.

A third example might be the encapsulation of the cyanine dye inside enzymically-degradable dendrimers. Dendrimers assembled with certain polyesters may be degraded by enzymes such as poly(3-hydroxybutyrate)-depolymerase or a catalytic antibody such as 38C2 catalyzing a retro-aldol process upon dendritic modified aliphatic polyesters. The degraded dendrimer then releases the trapped cyanine which can then assemble upon a suitable chem-bio-polymer/helical scaffold resulting in fluorescence turning “on” signaling the catalytic event of the enzyme or the antibody. Even non-catalytic events may be detected via the release of trapped or encapsulated cyanine. An example is the release of cyanine trapped within a photocleavable liposome following exposure to ultra-violet light.

Another example of a non-catalytic degradation of encapsulated cyanines might be the lability of vesicles under the influence of a pH change. For example, vesicles might be synthesized that are stable at low pH (such as those encountered in the gastric juice of the digestive system) but then disintegrate when the pH is raised to neutral. Such pH shifts might release the trapped/encapsulated cyanine dye which then can assemble upon chem-bio scaffolds resulting in an optical switch being turned “on” that signals the pH shift event. Biocompatible polymeric nanoparticles and molecular bio-capsules may also be prepared that encapsulate the cyanine and are released under a physiological or pahtological trigger such as apoptosis.

The released cyanine 92 may then assemble into helices with the appropriate biopolymers 94, resulting in the formation of J-aggregates 96, an increase in fluorescence and a shift in the absorption spectra.

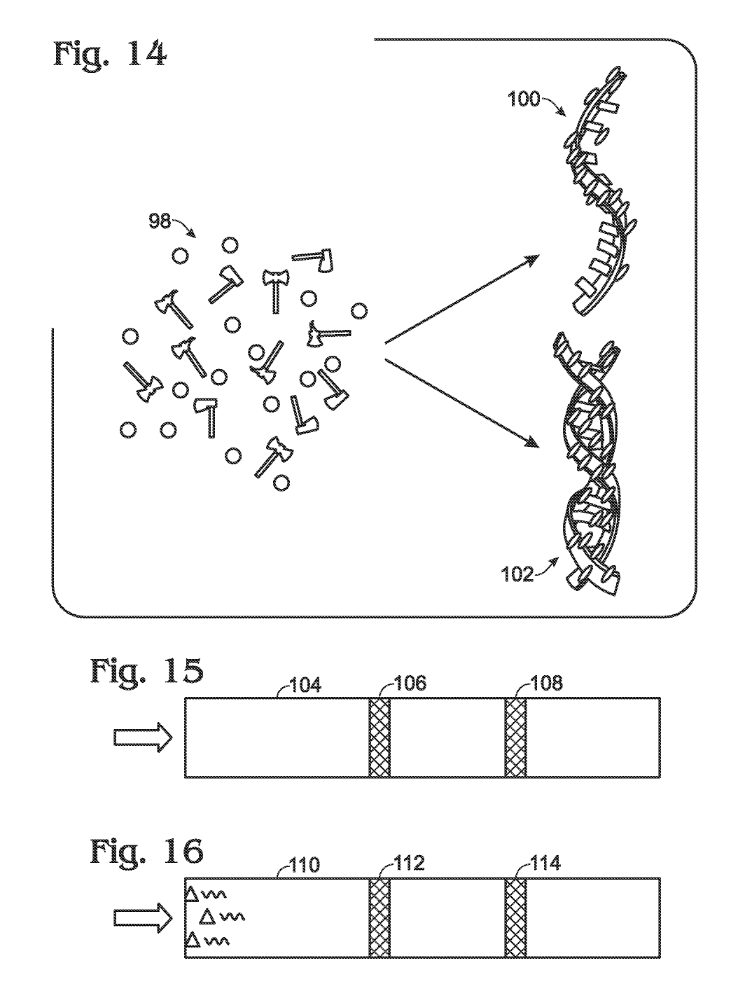

In a further embodiment, scaffold formation may take place using polymerase as shown in FIG. 14. Individual nucleotides are not sufficient to form a cyanine dye:biopolymer complex that forms the characteristic shift of the J-aggregate emissions. However, individual nucleotides may still bind to cyanine dyes, as shown at. The addition of a polymerase and the formation of oligomers of nucleotide:cyanine dye conjugates 100 (Oligomer with J-aggergated dye) and 102 (DNA Double helix with J-aggregated cyanine dye) would create the characteristic shift in the J-aggregate emissions resulting in a “turn-on” assay.

Scaffold formation assays may take any form applicable. An exemplary assay using tethered or otherwise isolated cyanine dye is shown in FIG. 15. In FIG. 15, a sample containing an analyte of interest is incubated with tethered or encapsulated cyanine dye (as described above). The reaction mixture is then introduced to a solid phase support 104. If the sample contains the analyte of interest, the cyanine dye will be released. In some embodiments, additional enzymes may be added to release the cyanine dye. The tethering or encapsulation compound(s) may be trapped in a first capture zone 106 while the cyanine dye continues to a second capture zone 108 containing molecules capable of forming chem-bio helices with cyanine dye and/or producing the spectral changes associated with J-aggregate formation. In some embodiments, such molecules may include, but are not limited to, clay nanoparticles, DNA or RNA duplexes, or other biopolymers such as linear polysaccharides such as cellulose, including carboxymethylamylose (CMA) and carboxymethylcellulose (CMC), and glycosaminoglycans; DNA; RNA; and proteins or structures composed of proteins such as albumin, Bacillus collagen-like antigen, cell membranes, phospholipids bilayers, liposomes, reverse micelles, gelatin, and pathophysiologically vital proteins including, but not limited to, potassium channel displaying extensive helices or the Streptococcal phage-encoded virulence factor.

In another embodiment, the scaffold formation assay may be a Lateral Flow Immunoassay (LFIA) as shown in FIG. 16. In LFIAs, a sample potentially containing a biopolymer of interest is introduced to a solid phase support containing one or more capture zones. Capture zones may include one or more test zones 112 which contain antibodies or other recognition molecules specific for the biopolymer of interest, and one or more control zones containing molecules capable of forming chem-bio helices with cyanine dye and/or producing the spectral changes associated with J-aggregate formation, or any other capture zone appropriate for the assay. In some embodiments, the control zone 114 may contain clay nanoparticles, DNA or RNA duplexes, or other biopolymers such as, but not limited to, linear polysaccharides such as cellulose, including carboxymethylamylose (CMA) and carboxymethylcellulose (CMC), and glycosaminoglycans; DNA; RNA; and proteins or structures composed of proteins such as albumin, Bacillus collagen-like antigen, cell membranes, phospholipids bilayers, liposomes, reverse micelles and gelatin. If the sample contains the biopolymer recognized by the antibody or recognition molecule immobilized in the test zone, the biopolymer will be captured. Cyanine dye may then be introduced to the solid phase support. If the sample contains the biopolymer of interest, the cyanine dye will bind in both the control zone and the test zone, producing characteristic absorption and fluorescence emissions. If the addition of the cyanine dye only produces an emission in the control zone then the biopolymer of interest is either not present or present at undetectable levels.

In some embodiments, the LFIA may be conducted as a sandwich assay in which a second reagent is added to the solid phase support after the test sample is introduced. In the sandwich assay, there may be one or more capture zones including at least one test zone containing antibodies or recognition molecules specific for the analyte of interest and one or more control zones containing molecules capable of forming chem-bio helices with cyanine dye and/or producing the spectral changes associated with J-aggregate formation.

The sample containing the analyte of interest is introduced to the solid phase support and binds to the test zone. A second reagent containing a biopolymer capable of binding to cyanine dye and forming J-aggregates and capable of binding to the analyte of interest or coupled to another molecule capable of binding the analyte of interest is then introduced to the solid phase support. Once the second biopolymer has bound to the captured biopolymer of interest, cyanine dye may be introduced. If the analyte of interest is present, the cyanine dye will bind to both the biopolymer attached to the analyte of interest and the biopolymer in the control zone yielding a positive result. This permits the detection of both helical biopolymers and non-helical or other small molecular mass analytes that do not bind to cyanine or cause J-aggregation.

In another embodiment, the LFIA assay may be a displacement assay. In a standard displacement assay, a sample flows through a membrane having binding elements with binding sites saturated with a labeled form of the analyte. The analyte in the sample displaces, under non-equilibrium conditions, the labeled form of the analyte from the membrane. The displaced labeled form of the analyte may then be detected. In the present invention, it may not be necessary to use a labeled form of the analyte though labeled forms may also be used. For example, a biopolymer that reacts with cyanine dye may be weakly bound to a solid phase support in an additional capture zone such as a binding zone. A sample potentially containing a biopolymer of interest may then be added to the solid phase support. The biopolymer of interest displaces the weakly bound biopolymer, washing it downstream to a second capture zone where it may be immobilized by an antibody or other recognition molecule. The addition of cyanine dye to the solid phase support may generate J-aggregation fluorescence in the binding zone, the second capture zone or a control zone. Emissions in the second capture zone and control zone indicate a positive test for the analyte of interest. Emissions in only the control zone would indicate a negative test result.

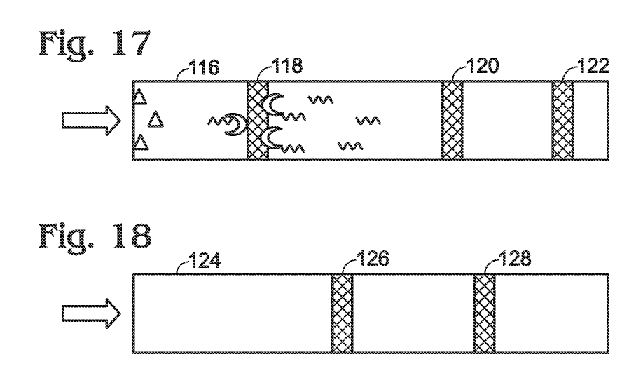

Assays may further comprise lateral flow displacement assays (LFDA). An exemplary LFDA is shown in FIG. 17. In a LFDA, a sample potentially containing a biopolymer of interest may be added to a solid phase support 116 containing one or more capture zones. Capture zones may include one or more recognition zones 118 which may contain samples of a biopolymer and/or biopolymer:cyanine dye complexes bound by antibodies or other recognition elements that have a higher affinity for the biopolymer of interest than the biopolymer and/or biopolymer:cyanine dye complexes, test zones 120 which contain antibodies or other affinity ligand to the biopolymer or biopolymer:cyanine dye complexes and one or more control zones which may contain molecules capable of forming chem-bio helices with cyanine dye and/or producing the spectral changes associated with J-aggregate formation, or any other capture zone appropriate for the assay. In some embodiments, the control zone 122 may contain clay nanoparticles, DNA or RNA duplexes, other biopolymers such as, but not limited to, linear polysaccharides such as cellulose, including carboxymethylamylose (CMA) and carboxymethylcellulose (CMC), and glycosaminoglycans; DNA; RNA; and proteins or structures composed of proteins such as albumin, Bacillus collagen-like antigen, cell membranes, phospholipids bilayers, liposomes, reverse micelles, gelatin, and pathophysiologically vital proteins including, but not limited to, potassium channel displaying extensive helices or the Streptococcal phage-encoded virulence factor or any other appropriate molecule.

If the biopolymer of interest is present in the sample, it will displace the biopolymer or biopolymer:cyanine dye complex from the recognition zone. The displaced biopolymer or biopolymer:cyanine dye complex then travels downstream to a second capture zone, the test zone. Cyanine dye may then be added to the solid support. If the original antibody captured molecule already has a cyanine dye, the strip 118 will be colored before the test and the color will be depleted as the cyanine-biopolymer-complex is “displaced” by the analyte. If there is no dye in the system to begin with, dye may be captured at all three sites (118, 120 and 122) depending on the affinity of the bound analyte complex for the cyanine. Accordingly, the recognition zone might produce characteristic emissions but it also may be tuned so that it does not.

In a further embodiment, the assay may be a lateral flow without PCR (LFWP) assay. An exemplary LFWP is shown in FIG. 18. In a LFWP, a test sample potentially containing DNA of interest may be added to a solid phase substrate 124 containing one or more capture zones. Capture zones may include, but are not limited to, one or more test zones 126 containing immobilized DNA strands that are in whole or in part complimentary to the DNA of interest, one or more control zones containing molecules capable of forming chem-bio helices with cyanine dye and/or producing the spectral changes associated with J-aggregate formation, or any other capture zone appropriate for the assay. In some embodiments, the control zone 128 may contain clay nanoparticles, DNA or RNA duplexes, other biopolymers such as, but not limited to, linear polysaccharides such as cellulose, including carboxymethylamylose (CMA) and carboxymethylcellulose (CMC), and glycosaminoglycans; DNA; RNA; and proteins or structures composed of proteins such as albumin, Bacillus collagen-like antigen, cell membranes, phospholipids bilayers, liposomes, reverse micelles and gelatin or any other appropriate molecule.

If the sample contains the target DNA, it hybridizes with the immobilized DNA strands. Addition of cyanine dye to the solid phase support will result in the cyanine dye intercalcating with the duplex DNA in the test zone to produce emission and absorption spectral changes. In the absence of the DNA of interest, only the control zone will produce the expected emission and absorption changes.

In some embodiments, biotinylated oligonucleotides may be added to the solid phase support prior to the addition of the cyanine dye. These oligonucleotides have short DNA sequences that are complementary to regions of the target DNA. After the oligonucleotides have bound to the captured DNA, streptavidin or other biotin binding fragment conjugated biopolymer is added to the solid phase support. The addition of cyanine dye to the complementary-DNA-target-DNA-oligonucleotide-biotin-streptavidin-biopolymer complex will produce the characteristic absorption and fluorescence changes indicative of the formation of J-aggregates in the test zone indicating the presence of the DNA of interest.

Changes in fluorescence and emission spectra in the assays described above may be read by any appropriate commercially available reader or scanner as well as specifically designed readers or scanners. In one embodiment, such units may be bench-top or portable field instruments with an optical source, lamp and other electronics to facilitate measurements of light absorption and fluorescence emission. Such readers or scanners may be powered by any means applicable including direct current, wall-plug or batteries. In one embodiment, the reader or scanner used may be capable of exciting radiation from ultra-violet (UV) through the entire visible light spectra. For example, such a reader will be capable of exciting radiation in a range of about 220 nm to 750 nm, more specifically from about 220 nm to 700 nm, 300 to 600 nm, or from about 350 to 540 nm. In another embodiment, the reader or scanner used may be capable of detecting fluorescence or luminescence emission light wavelengths over the ultraviolet through visible light spectrum. For example, such a reader will be capable of detecting fluorescence in a range of about 220 nm to 750 nm, more specifically from about 220 nm to 700 nm, 300 to 600 nm, or from about 350 to 540 nm.

The electronic reader or scanner may be able to read a variety of assay platforms including, but not limited to, beads, microparticles, tubes, fabrics, plates, latex particles, magnetic particles, paper, dipsticks, nano-particles, coupons, or tickets, formed from or coated with these materials as well as alternative flow-based formats such as channels and multiplexed assays on patterned substrates and the like and will have slots of the appropriate shape and size to accommodate such platforms.

In some embodiments, the electronic reader or scanner may be capable of measuring changes intensities of light absorption or fluorescence emission as well as wavelengths (spectral) shifts. Such measurements may be outputted as raw data, or relative comparisons such as current strengths of light signals, relative fluorescence units, or counts per second, or any other appropriate output units.

In other embodiments, the electronic reader or scanner may be integrated either through hardwiring or wirelessly to remote stations where the data may be downloaded and analyzed. Appropriate software may be used to process the data and/or to facilitate minimal intervention with the instrument on the part of the processor. In some embodiments, the electronic reader or scanner may have charting or graphing capabilities as well as visual and audible indications of test results.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a channel” includes a plurality (for example, a culture or population) of such channels, and so forth.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein.

### EXAMPLES

The examples below provide evidence of novel label-free screening assays using cyanine dyes in order to identify enzyme activity and specific biopolymers. Such assays may be used in either a “turn on” or “turn off” mode increasing or decreasing the emissions spectra of the cyanine dyes.

### Example I

### Collection of Amylase

Human saliva was collected from a volunteer and then centrifuged at 14000 rpm (16000×g) for one minute at ambient temperature (25° C.), using a Galaxy 16D microcentrifuge (VWR International, West Chester, Pa.). The resulting supernatant was collected and re-centrifuged as described above. The supernatant from the second centrifugation was centrifuged again as described above and the supernatant from the third centrifugation was used as the source of amylase.

### Example II

### Amylase Assay as a Function of Time

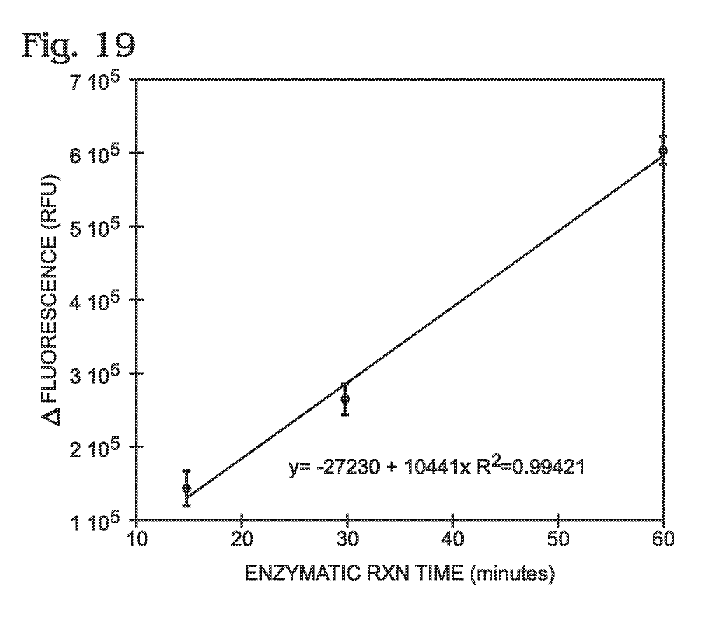

75 μM of carboxymethylamylose (CMA) was mixed with 5 μL of saliva in a total volume of 60 μL water and incubated at ambient temperature (25° C.). At 0, 10, 20, 30, 40, 50, and 60 minutes, one half of the reaction volume was removed and added to 270 μL of a 7.5 μM solution of cyanine of Formula I dissolved in 20% methanol-80% water mixture and dispensed into the wells of a 96 well microplate (Optiplate™-96, Perkin Elmer Life Sciences, Boston, Mass.). Fluorescence from the resulting J-aggregate was then immediately measured using a Mithras LB 940 microplate spectrofluorometer (Berthold Instruments, Oak Ridge, Tenn.). As can be seen in FIG. 19, a linear (r²=0.994) increase in relative fluorescence units (RFU, A of fluorescence was obtained with increasing reaction time). RFU=relative fluorescence unit; Delta, i.e. Δ RFU, denotes the difference in fluorescence intensity in the presence and basence of the signaling event. The signaling event in this case might be the self-assembly of the cyanine upon a suitable chem-bio-polymer resulting in fluorescence emission or the degradation of the scaffold resulting in diminished fluorescence intensity

### Example III

### Amylase Assay as a Function of Concentration

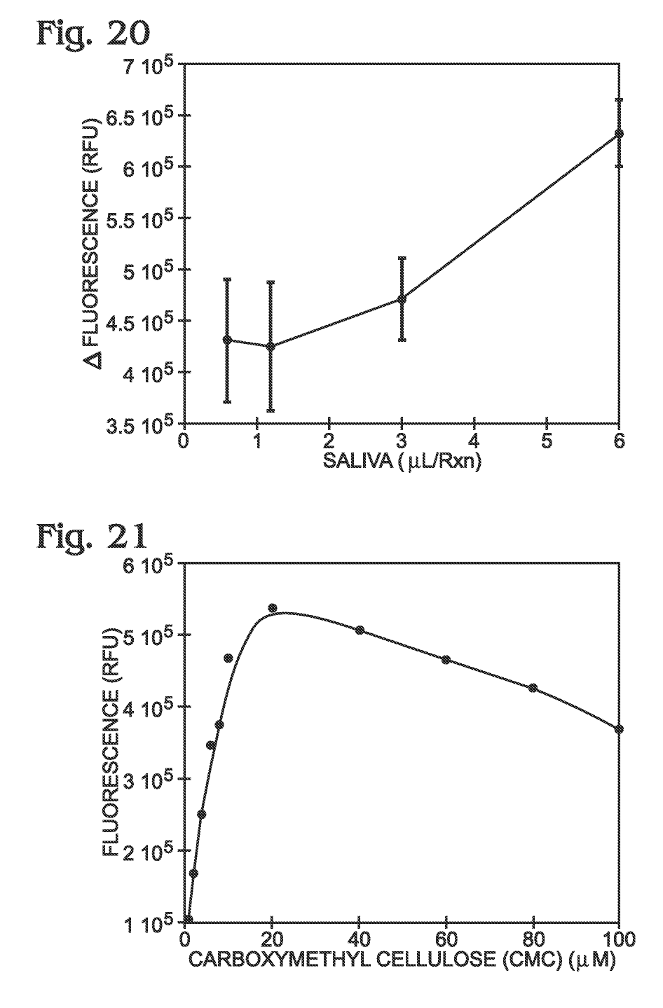

75 μM of carboxymethylamylose (CMA) was mixed with 0, 1, 2, 3, 4, 5 and 6 μL of saliva in a total volume of 60 μL water and incubated at ambient temperature (˜25° C.). After 60 minutes, one half of the reaction volume for each respective combination was removed and added to 270 μL of 7.5 μM cyanine dye of Formula I dissolved in 20% methanol-80% water mixture dispensed into the wells of a 96 well microplate (Optiplate™-96, Perkin Elmer Life Sciences, Boston, Mass.). Fluorescence from the formation of J-aggregates in the sample was then immediately measured using a Mithras LB 940 microplate spectrofluorometer (Berthold Instruments, Oak Ridge, Tenn.). As can be seen in FIG. 20, the greater the concentration of amylase, the greater the fluorescence intensity indicating enzyme-mediated destruction of the CMA scaffold. The decrease in fluorescence of the cyanine dye:CMA J-aggregate for a constant incubation time shows a dependence on the estimated amylase concentration.

### Example IV

### Carboxymethyl Cellulose Titration

Carboxymethylcellulose (CMC) in 0, 20, 40, 60, 80 and 100 μM concentrations was added to the wells of a 96-well microplate containing 10 μM of cyanine dissolved in 20% methanol-80% water mixture and was dispensed into the wells of a 96 well microplate (Optiplate™-96, Perkin Elmer Life Sciences, Boston, Mass.) to a total volume of 275 μL. Fluorescence was immediately measured using a Mithras LB 940 microplate spectrofluorometer (Berthold Instruments, Oak Ridge, Tenn.). As can be seen in FIG. 21, optimal fluorescence emission was observed at 20 μM CMC polymer repeat units (or 17.4 nM CMC intact polymer) against a fixed concentration of 10 μM cyanine dye of Formula I. At 10 μM cyanine dye and 10 μM CMC, the fluorescence emission intensity reached 87% of maximum. The estimated limits of detection and quantitation for CMC are 0.3 μM and 1.0 μM respectively for the polymer repeat unit. For intact CMC polymer the limits of diction and quantitation are estimated to be 291 pM and 872 pM respectively.

### Example V

### Cyanine Dye Titration with Carboxymethylcellulose

Cyanine dye of Formula I dissolved in 20% methanol-80% water was added to wells of a 96-well microplate (Berthold Instruments, Oak Ridge, Tenn.) in concentrations of 0, 20, 40, 60, 80 and 100 μM. Then, either 10, 20, or 40 μM of carboxymethylcellulose was added to the various concentrations of cyanine dye to reach a total volume in each microwell of 275 μL. Fluorescence emission was measured immediately after the addition of the carbodymethylcellulose (CMC) using a Mithras LB 940 microplate spectrofluorometer (Berthold Instruments, Oak Ridge, Tenn.). As can be seen in FIG. 22, optimal fluorescence emission was again observed between 0.5 to 1 and 1 to 1 ratio of polymer repeat units to the dye. Only a small increase in fluorescence emission was observed between 20 μM and 40 μM CMC at the optimal 40 μM cyanine dye concentrations. At sub-optimal concentrations of the cyanine dye of less than 20 μM, all three CMC concentrations of 10, 20 or 40 μM were adequate to provide scaffolding, resulting in nearly identical fluorescence emission intensities. It is estimated that the limits of detection and quantitation for cyanine dye using CMC as a scaffold are 3 μM and 5 μM respectively.

### Example VI

### Hyaluronic Acid Titration Against a Fixed Concentration of Cyanine Dye

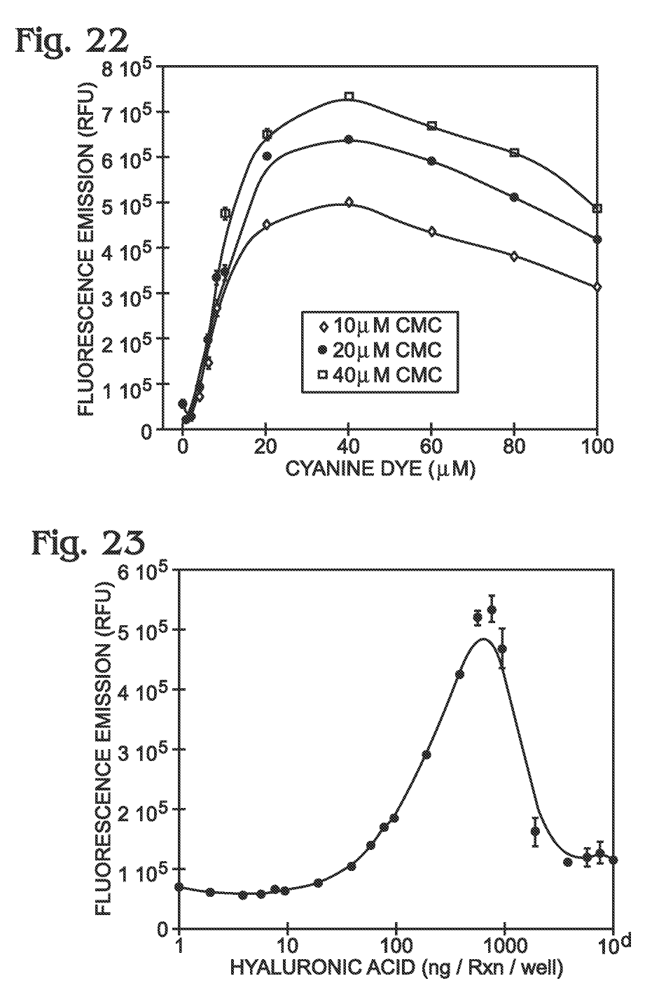

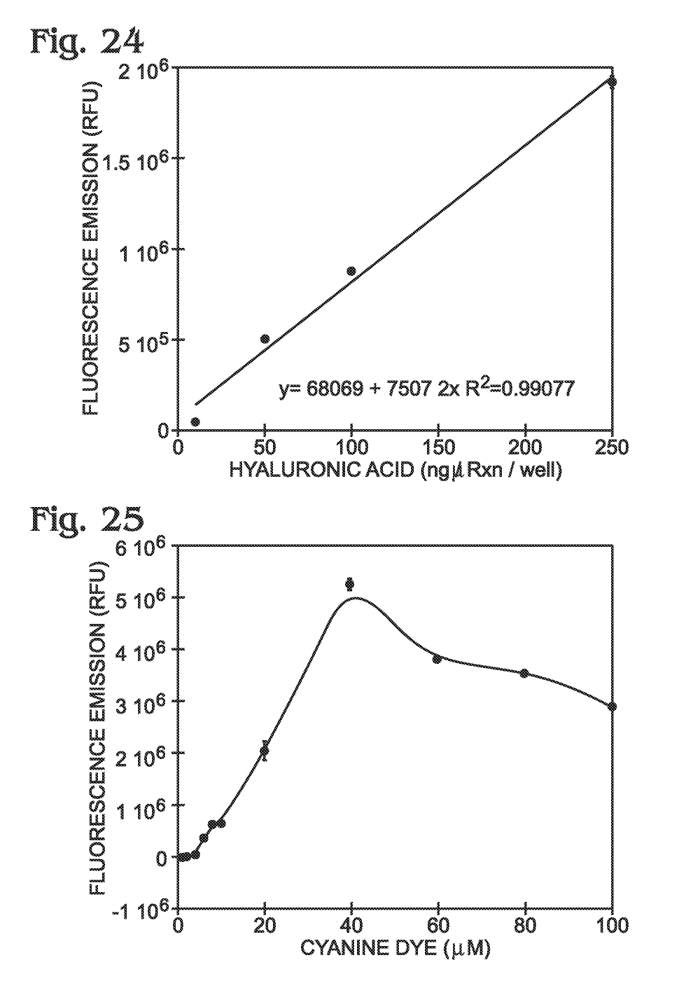

Hyaluronic acid in concentrations of 1, 10, 100, 1000 or 10,000 ng was added to 20 μM concentrations of cyanine dye of Formula I dissolved in a 20% methanol-80% water mixture and dispensed into the wells of a 96 well microplate (Optiplate™-96, Perkin Elmer Life Sciences, Boston, Mass.) for a total volume of 300 μL. Fluorescence emission was measured after 60 minutes of incubation at 25° C. using a Mithras LB 940 microplate spectrofluorometer (Berthold Instruments, Oak Ridge, Tenn.). As can be seen in FIG. 23, titrating increasing amounts of hyaluronic acid against a fixed concentration of cyanine dye resulted in a bell shaped curve for fluorescence intensity. As shown in FIG. 24, fluorescence emission was linear (r²=0.991) between 10 and 250 ng of hyaluronic acid yielding a limit of detection value of ˜40 pM; or ˜10 fmol HA/300 μL of reaction volume in a 96 well microplate.

### Example VII

### Cyanine Dye Concentration Against a Fixed Concentration of Hyaluronic Acid

Concentrations of 0, 20, 40, 80 and 100 μM cyanine dye cyanine dissolved in 20% methanol-80% water mixture was dispensed into the wells of a 96 well microplate (Optiplate™-96, Perkin Elmer Life Sciences, Boston, Mass.). Then, 1.0 μg of hyaluronic acid was added to each well. The total volume inside each microwell was 275 μL. Fluorescence was measured 60 minutes after the addition of hyaluronic acid using a Mithras LB 940 microplate spectrofluorometer (Berthold Instruments, Oak Ridge, Tenn.). As can be seen in FIG. 25, 40 μM of cyanine dye produced the maximal fluorescence emission when combined with 1.0 μg of hyaluronic acid.

### Example VIII

### Scaffold Disruption/Destruction Fluorescent Assay

Hyaluronidase enzyme (11.3 U of E.C. 3.2.1.35) was mixed with 8 μg of hyaluronic acid in a total volume of 500 μL at 25° C. After 0, 20, 40, 60, 80, 100, 120 and 140 minutes, a portion of the reaction mixture was diluted into 20 μM concentrations of cyanine dye of Formula I dissolved in 20% methanol-80% water mixture dispensed into the wells of a 96 well microplate (Optiplate™-96, Perkin Elmer Life Sciences, Boston, Mass.) to a total volume of 275 μL. Sixty minutes after adding the reaction mixture to the cyanine solution, the fluorescence was measured using a Mithras LB 940 microplate spectrofluorometer (Berthold Instruments, Oak Ridge, Tenn.).

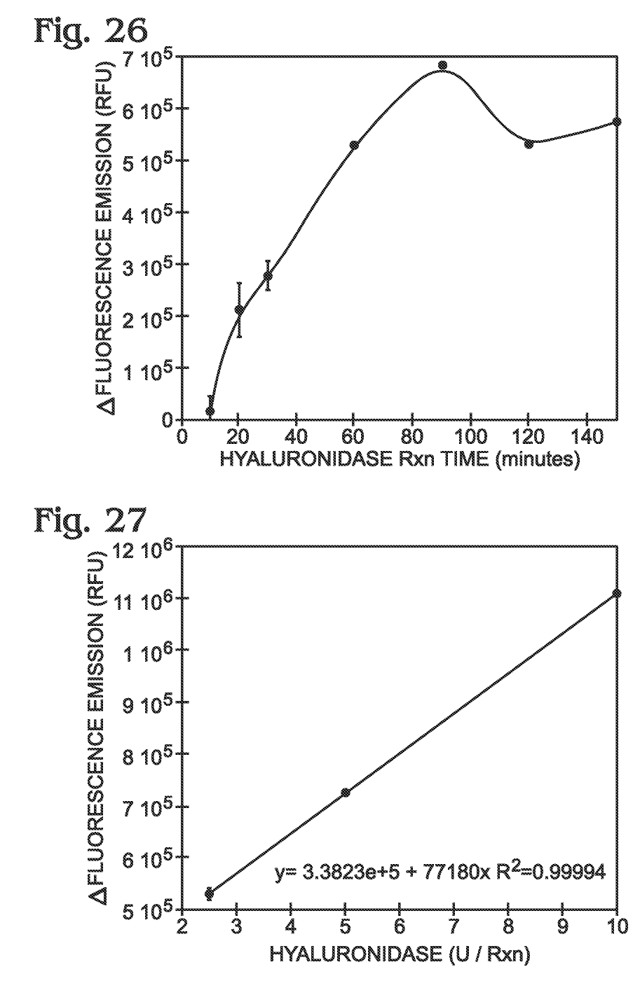

The enzymatic reaction volume that was added to the cyanine dye solution represented approximately 364 ng of intact hyaluronic acid that might be present in the complete absence of enzymatic hydrolysis. This amount of hyaluronic was equivalent to the mid-point in the rise of the hyaluronic titration curve in FIG. 23. As can be seen in FIG. 26 the assay functions as a “turn off” assay, based on the phenomenon of “scaffold destruction/disruption.

### Example IX

Varying concentrations of hyaluronidase enzyme of 2, 3, 4, 5, 6, 7, 8, 9, and 10 U respectively were mixed with 10 μg of hyaluronic acid in a total reaction volume of 500 μl. After 180 minutes at 25° C., a portion of the reaction mixture was diluted into 20 μM concentrations of cyanine dye of Formula I dissolved in 20% methanol-80% water mixture and was dispensed into the wells of a 96 well microplate (Optiplate™-96, Perkin Elmer Life Sciences, Boston, Mass.) to a total volume of 275 μL.

Fluorescence was measured 60 minutes after the addition of the enzymatic reaction mixture to the cyanine dye solution using a Mithras LB 940 microplate spectrofluorometer (Berthold Instruments, Oak Ridge, Tenn.). As can be seen in FIG. 27, there was a linear (r²=0.999) increase in enzymatic activity with increasing concentrations of the hyaluronidase enzyme.

### Example X

### Dose-Response Behavior of Hyaluronidase Activity

Hyaluronic acid in amounts of 0, 200, 400, 600, 800 and 1000 ng respectively were mixed with 1 U of hyaluronidase in a total volume of 100 μL. After allowing the enzymatic reaction to proceed for 240 minutes at 25° C., a portion of the reaction mixture equivalent to approximately 364 ng of intact HA that might be present in the complete absence of enzymatic hydrolysis was added to the wells of a 96 well microplate (Optiplate™-96, Perkin Elmer Life Sciences, Boston, Mass.) containing 20 μM concentrations of cyanine dye of Formula I dissolved in a 20% methanol-80% water mixture to a total volume of 275 μL.

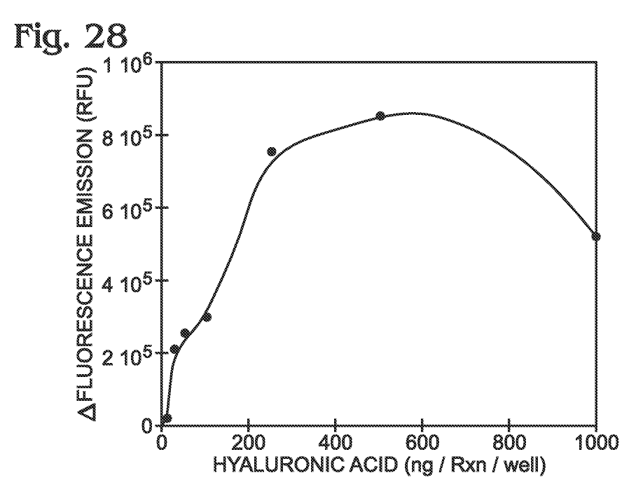

The dose response behavior of the hyaluronidase activity to increasing concentration of the hyaluronic acid substrate is shown in FIG. 28. As can be seen in FIG. 28, measurable enzymatic activity was detected with 15 to 20 minutes of catalysis. Based upon maximal fluorescence from reactions in which the enzyme and substrate were mixed but not allowed to react, it was estimated that hyaluronidase activity level reached a plateau between 60 to 80 minutes, representing 50% hydrolysis of the substrate.

Although the foregoing invention has been described in detail by way of example for purposes of clarity of understanding, it will be apparent to the artisan that certain changes and modifications may be practiced within the scope of the appended claims which are presented by way of illustration not limitation. In this context it will be understood that this invention is not limited to the particular formulations, process steps, and materials disclosed herein as such formulations, process steps, and materials may vary somewhat. It will also be understood that the terminology employed herein is used for the purpose of describing particular embodiments only, and is not intended to be limiting since the scope of the present invention will be limited only by the appended claims and equivalents thereof. It is further noted that various publications and other reference information have been cited within the foregoing disclosure and listed in the reference section below for economy of description. Each of these references are incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

We claim:

1. A method comprising:
(a) providing a fluorescent dye;
(b) providing a biopolymer;
(c) providing a sample suspected of containing an analyte of interest, wherein the analyte, if present, is capable of inducing a conformational change in the biopolymer;
(d) forming a reaction mixture comprising the sample, the fluorescent dye, and the biopolymer; and
(e) measuring the emission spectra of the reaction mixture.

2. The method of claim 1, wherein the analyte of interest is an enzyme.

3. The method of claim 1, wherein the fluorescent dye is a cyanine dye.

4. The method of claim 1, wherein the cyanine dye is the cyanine dye of Formula I:

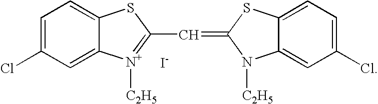

5. The method of claim 1 further comprising exposing the biopolymer to the fluorescent dye under suitable conditions to form a fluorescent dye:biopolymer complex and wherein the formed fluorescent dye:biopolymer complex is then exposed to the sample.

6. The method of claim 5 wherein the fluorescent dye:biopolymer complex has a known emission spectra.

7. The method of claim 6 further comprising comparing the emission spectra of the reaction mixture with the known emission spectra of the fluorescent dye:biopolymer complex to determine the presence and concentration of the analyte.

8. The method of claim 5, wherein the fluorescent dye:biopolymer complex forms J-aggregates.

9. The method of 1 wherein providing a biopolymer comprises the steps of:
(a) providing a substance that is capable of forming a biopolymer in the presence of the analyte of interest; and
(b) exposing the substance to the sample so that a biopolymer is formed if the analyte is present in the sample.

10. The method of claim 9 further comprising exposing the formed biopolymer to the fluorescent dye.

11. The method of claim 1 wherein the cyanine dye is encapsulated.

12. The method of claim 1 wherein at least one of the fluorescent dye, biopolymer, or sample is provided on a solid phase support.

13. The method of claim 12 wherein the solid phase support comprises a capture region configured to trap released fluorescent dye.

14. The method of claim 12 wherein the solid phase support comprises a reaction zone comprising recognition molecules specific to the analyte of interest.

15. A method for detecting the presence of an analyte comprising:
(a) combing a sample suspected of containing an analyte with a fluorescent dye to form a reaction mixture;
(b) measuring the emission spectra of the reaction mixture;
(c) adding an enzyme that degrades the suspected analyte to the reaction mixture;
(d) measuring the emission spectra of the reaction mixture containing the enzyme;
(e) comparing the emission spectra of the reaction mixture containing the enzyme with the reaction mixture of the fluorescent dye and the sample; and
(f) determining if the emission spectra has altered.

16. The method of claim 12, wherein the fluorescent dye is a cyanine dye.

17. The method of claim 12, wherein the fluorescent dye and the analyte form J-aggregates.

18. The method of claim 12, wherein the sample contains a plurality of analytes.

19. A method for detecting the presence of an analyte in a sample comprising:
(a) contacting the analyte with a specific binding partner for said analyte under conditions suitable to cause the binding of at least a portion of said analyte to said specific binding partner to form a complex;
(b) binding a biopolymer capable of forming J-aggregates with cyanine dye to a second binding partner for said analyte under conditions suitable to cause the binding of at least a portion of said second binding partner to the biopolymer;
(c) combining the analyte with a specific binding partner with the biopolymer with a second binding partner to form a first mixture;
(d) adding cyanine dye to the first mixture to form a second mixture;
(e) measuring the emissions of the J-aggregates in the second mixture; and
(f) correlating the emitted fluorescence with the presence or the amount of said analyte in said sample.

20. The method of claim 19 wherein at least one of the first or second binding partners is attached to a solid support.
